(19) United States
(12) Patent Application Publication
(10) Pub. No.: US 2005/0215870 A1
(43) Pub. Date: Sep. 29, 2005

(54) System for monitoring physical parameters of individuals performing physical activities and apparatus for indicating oar positions

(76) Inventor: William B. Rademaker, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/084,856

(22) Filed: Mar. 18, 2005

Related U.S. Application Data
(60) Provisional application No. 60/554,708, filed on Mar. 19, 2004.

(51) Int. Cl. A61B 5/00; A61B 5/02; A61B 5/08

(52) U.S. Cl. 600/301; 128/903; 600/549; 600/485; 600/500; 600/529; 128/905

(57) ABSTRACT

In one aspect, the invention provides systems for monitoring at least one physical parameter of individuals engaging in a team sport, such as rowing. Another aspect of the invention provides apparatuses for indicating oar position and methods for improving rowing techniques.

### CROSS-REFERENCE TO RELATED APPLICATION

This application claims the benefit of U.S. Provisional Application No. 60/554,708, filed Mar. 19, 2004, under 35 U.S.C. § 119.

### FIELD OF THE INVENTION

The present invention relates generally to systems and methods for simultaneously monitoring at least one physiological parameter and/or performance parameter of a plurality of individuals engaged in a physical activity such as a team sport, and apparatuses for indicating oar positions.

### SUMMARY OF THE INVENTION

One aspect of the invention provides systems and methods for monitoring at least one physical parameter of individuals engaging in a physical activity, such as a team sport. In some embodiments, the system comprises (a) a plurality of sensors, wherein each sensor is operatively coupled to one of a plurality of individuals engaging in a team sport, wherein each sensor is adapted to monitor and transmit information about at least one physical parameter of one of the plurality of individuals, and wherein the physical parameter is at least one of a physiological parameter and a performance parameter; and (b) a receiver adapted to receive and display the transmitted information from each of the plurality of sensors. The system of the invention is useful for monitoring one or more physical parameters of each of a plurality of individuals engaging in any team sport, including, but not limited to, rowing, skiing, football, basketball, and bicycling. The physical parameter may be a physiological parameter or a force output parameter.

In some embodiments, the plurality of sensors comprises sensors adapted for monitoring a physiological parameter selected from the group consisting of heart rate, temperature, breathing rate, perspiration rate, blood pressure, and lactic acid levels. In some embodiments, the plurality of sensors comprises sensors adapted to monitor a performance parameter selected from the group consisting of stress placed on an oar by an individual, load placed on an oar against a boat, and load placed on a boat by an individual. The plurality of sensors may also comprise both sensors adapted to monitor a performance parameter and sensors adapted for monitoring a physiological parameter from each of the plurality of individuals.

In some embodiments, the methods for monitoring at least one physical parameter of individuals engaging in a team sport, comprising the steps of (a) operatively coupling a plurality of sensors to a plurality of individuals engaging in a team sport, wherein each sensor is operatively coupled to one of the plurality of individuals, wherein each sensor is adapted to monitor and transmit information about at least one physical parameter of one of the plurality of individuals to a receiver adapted to receive and display information from the plurality of sensors, and wherein the physical parameter is at least one of a physiological parameter and a performance parameter; and (b) monitoring at least one physical parameter of each of the plurality of individuals by observing the information displayed on the receiver. The methods may further comprise the step of providing feedback to at least one of the plurality of individuals.

A second aspect of the invention provides apparatuses for indicating the position of an oar during rowing and methods for improving an individual's stroke technique by using an apparatus for indicating the position of an oar during rowing. For example, an apparatus for indicating the position of an oar may be used to indicate desirable oar positions. In some embodiments, the apparatus for indicating oar position comprises a position indicator extending from a support assembly operatively coupled to a boat.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows an isometric view of athletes rowing a boat including physiological/performance sensors, transmitters and displays in accordance with an embodiment of the invention.

FIG. 2 shows an enlarged isometric view of an athlete rowing a boat of FIG. 1 and associated physiological/performance sensors, transmitters, and displays in accordance with an embodiment of the invention.

FIG. 3 shows an isometric view of a display unit in accordance with an embodiment of the invention.

FIG. 4 shows an isometric view of an athlete pulling an oar including physiological/performance sensors in accordance with an embodiment of the invention.

FIG. 5 shows an isometric view of an athlete pulling an oar including physiological/performance sensors in accordance with an embodiment of the invention.

FIG. 6 shows an isometric view of an athlete pulling an oar including physiological/performance sensors in accordance with an embodiment of the invention.

FIG. 7 shows an isometric view of a display unit in accordance with an embodiment of the invention.

FIG. 8 shows an isometric view of a crucial data comparison in accordance with an embodiment of the invention.

FIGS. 9A and 9B show an isometric view of a boat and oar and a position indicator in accordance with an embodiment of the invention.

FIG. 10 shows an isometric view of a sidewall of a boat and a position indicator attachment in accordance with an embodiment of the invention.

FIG. 11 shows an isometric view of a sidewall of a boat and a position indicator attachment in accordance with an embodiment of the invention.

FIG. 12 shows an isometric view of a sidewall of a boat and a position indicator attachment in accordance with an embodiment of the invention.

FIG. 13 shows an isometric view of a sidewall of a boat and a position indicator attachment in accordance with an embodiment of the invention.

### DETAILED DESCRIPTION OF THE PREFERRED EMBODIMENT

One aspect of the invention provides systems and methods for monitoring at least one physical parameter of individuals engaging in a physical activity, such as a team sport. In some embodiments, the system comprises (a) a plurality of sensors, wherein each sensor is operatively coupled to one of a plurality of individuals engaging in a team sport, wherein each sensor is adapted to monitor and transmit information about at least one physical parameter of one of the plurality of individuals, and wherein the physical parameter is at least one of a physiological parameter and a performance parameter; and (b) a receiver adapted to receive and display the transmitted information from each of the plurality of sensors. The system of the invention is useful for monitoring one or more physical parameters of each of a plurality of individuals engaging in any team sport, including, but not limited to, rowing, skiing, football, basketball, and bicycling. The physical parameter may be a physiological parameter or a force output parameter. Exemplary physiological parameters include, but are not limited to, heart rate, pulse, skin temperature, blood pressure, lactic acid levels, breathing rate, and perspiration rate. Exemplary performance parameters include, but are not limited to, force output parameters. For example, in a system for monitoring at least one physical parameter of individuals that are rowing, performance parameters may include force output parameters such as force/load placed on an oar by an individual, stress placed on an oar by an individual, load placed on/by an oar against a boat, and load placed on a boat by an individual; force/load placed on a pedal/foot-plate by an individual, force/load placed on a foot-stretcher by an individual, stress placed on a foot-stretcher by an individual, stress placed on a bat/club/pole by an individual, and force/load placed on a bat/club/pole by an individual.

In some embodiments, the invention provides systems for monitoring at least one physical parameter of a plurality of individuals engaged in rowing. The system may monitor a physiological parameter, a performance parameter, or both. For example, the system may monitor force/load output placed by a group of rowers on their respective oars during a rowing stroke or the force/load placed by a plurality of oars against the boat at a plurality of contact points and simultaneously monitor the heart rates of the group of rowers.

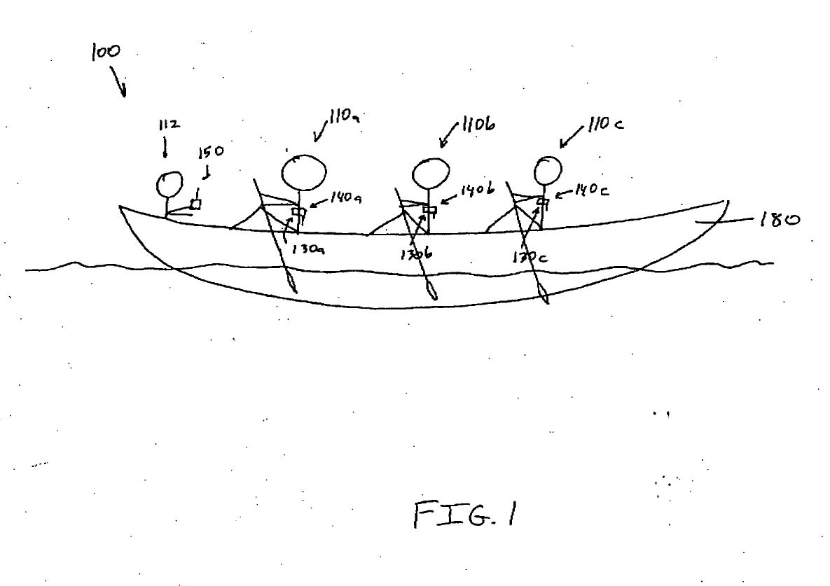

The following description with reference to FIGS. 1-8 illustrates exemplary embodiments of this aspect of the invention. However, one skilled in the art will understand that the invention may be practiced without these details. In other instances, well-known structures and functions have not been shown or described in detail to avoid unnecessarily obscuring the description of the embodiments of the invention.

Some embodiments of the invention provide a system for monitoring a physiological parameter of a plurality of individuals. FIG. 1 is a schematic side view of a system 100 for monitoring in real time the status of a physical characteristic of an individual or a group of individuals. The system 100 can include a plurality of sensors 130 (identified individually as 130a-c), a plurality of transmitters 140 (identified individually as 140a-c) operatively coupled to the sensors 130, and a receiver 150 to receive data from the transmitters 140. The system 100 may allow an individual such as a coach, or coxswain, 112 to monitor a physiological parameter of a plurality of athletes 110 (identified individually as 110a-c) who are rowing a boat 180, such as a sculling boat. Each athlete 110 has a sensor 130 that monitors the status of a physiological parameter of that particular athlete 110. In the illustrated embodiment, each athlete 110 also has a transmitter 140 that is electrically coupled to the sensor 130 to transmit data specific to the athlete 110 to the receiver 150. Accordingly, the coach 112 can monitor in real time the status of the physiological parameter for each athlete 110. In some embodiments, the system 100 may monitor a group of athletes or a single athlete engaged in biking, skiing, basketball, football, or many other sports or activities. Furthermore, the system 100 may monitor groups of individuals other than athletes.

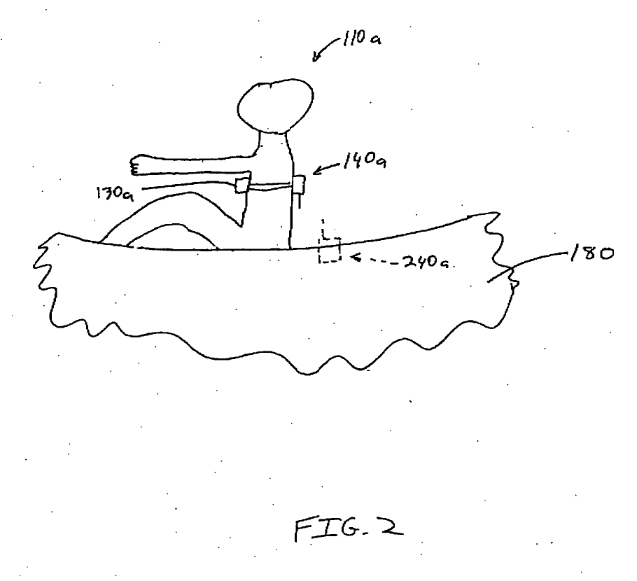

FIG. 2 is an enlarged schematic side view of one of the athletes 110 of FIG. 1. In the illustrated embodiment, the sensor 130a is positioned proximate to the heart of the athlete 110a to monitor the heart rate. The sensor 130a may be attached directly to the athlete's skin or worn over the athlete's clothing. In some embodiments, the sensor 130a may be positioned at other suitable locations on the athlete 110a to sense the heart rate. Suitable sensors 130 include, but are not limited to, heart rate monitors manufactured by Polar Electro Inc. of Woodbury, N.Y., or similar devices.

The system 100 may also monitor other physiological parameter of individuals. For example, the system 100 can monitor the skin temperature, blood pressure, lactic acid levels, breathing rate, perspiration rate, or the status of other physical characteristics. In some embodiments, multiple sensors may be placed on each individual to monitor the status of several parameters (physiological parameters or force output parameters) simultaneously.

In the embodiments shown in FIGS. 1 and 2, the transmitter 140a is worn by the athlete 110a and positioned proximate to the back of the athlete 110a so as not to interfere with the rowing motion. However, the transmitter 140a may be positioned at other suitable locations on the athlete 10a or proximate to the athlete 10a. For example, a transmitter 240a (shown in hidden lines) may be placed on the boat 180, or extension of the boat, proximate to the athlete 110a. As used herein, “extension of the boat” refers to any object connected or fastened to the boat, or fastened to another object that is, in turn, fastened to the boat. In additional embodiments, a single transmitter can receive data from multiple sensors 130 (FIG. 1) and transmit the data to the receiver 150. In any of these embodiments, the transmitters 140 may be electrically coupled to the sensors 130 to receive and transmit the status of a physiological parameter of an individual in real time.

The transmitters 140 may transmit data from the sensors 130 to the receiver 150 by radio frequency, infrared, or other wireless means. Accordingly, the coach 112 and the receiver 150 may be in the boat 180 with the athletes 110 or the coach 112 may be outside the boat 180 and monitor the status of the physiological parameter of the athletes 110 remotely. In some embodiments, the transmitters 140 may transmit data to the receiver 150 using a hardwired method. The data may also be transmitted using boosters. “Boosters” are defined as a device that captures a data stream or signal, and augments or re-directs the signal, for transmission to a receiver 150. Boosters may be positioned in the boat, for example, placed under each rower's seat. In addition, the transmitter may transmit data wirelessly to an on-board system, such as an on-board speaker system. For example, the data may be transmitted wirelessly initially, but would then be carried via a wire (either speaker wires already used in the boat or other wires), for example, to a computer such as an on-board coxswain computer. The term “on-board coxswain computer” or “cox-box” refers to a computer that measures time and stroke rating, and includes, but is not limited to, any current on-the-market computer used in rowing today, such as the rowing computer (CoxBox or Speed Coach) manufactured by Nielsen-Kellerman Co. of Boothwyn, Pa.

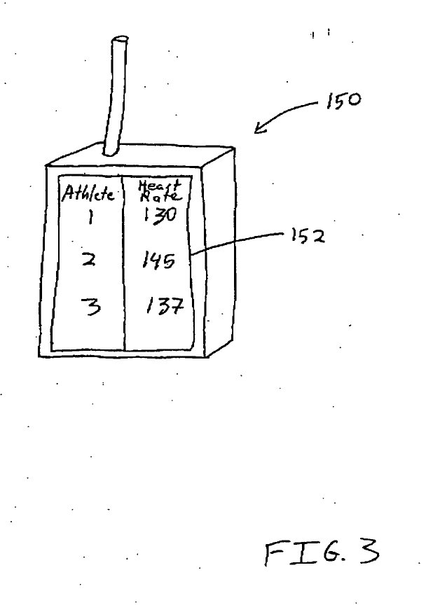

FIG. 3 shows a schematic isometric view of the receiver 150 of FIG. 1. The receiver 150 includes a display 152 indicating the status of each athlete's physical condition. For example, the display 152 may list the heart rate of each athlete 110. In some embodiments, the display 152 may provide other useful information, such as the change in the status of a physiological parameter over a period of time or a comparison between the present status of the physiological parameter and the status at a previous time. For example, the display 152 may list the present heart rate and the change in the heart rate over the last minute for each athlete.

The receiver 150 may be a hand-held device. However, receivers 150 that are not hand-held may also be used. For example, the receiver 150 may be coupled to a computer display, or coupled with, or wired directly to, the cox box. The receiver 150 may be integrated into, or combined with, the cox box, attached as a separate display, or not attached at all. Accordingly, the display 150 may be integrated into the display of the current cox box rowing computers on the market. In another embodiment, the receiver 150 may be a small device configured to be worn by an individual, such as a watch or a personal digital assistant-like display unit (e.g., a palm pilot). In some embodiments, the display 150 is attached to the boat proximate to the coxswain.

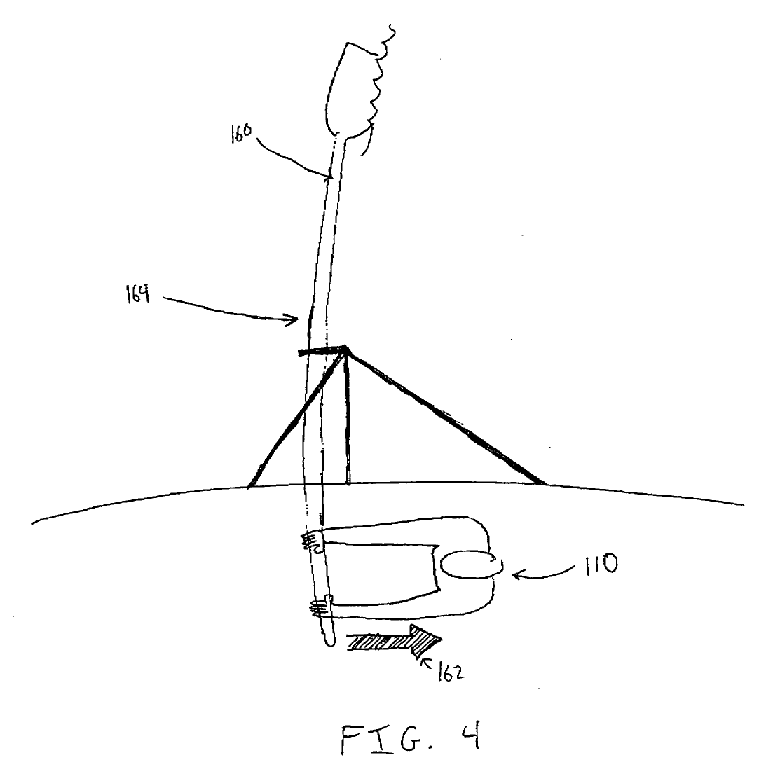

In some embodiments, the system may monitor aspects of the individuals' performance output. FIG. 4 is a schematic view of a system for monitoring the load placed by an athlete 110 on the oar 160. The system may monitor the load or stress placed on the oar 160 during the stroke. The force of the athlete 110 pulling, as indicated by 162, causes the oar 160 to experience stress and/or bend. A strain gauge 164 or other device may be used to measure the stress or strain in the oar 160 and may be attached to the oar 160. In some embodiments, a plurality of strain gauges 164 or other devices used to measure the load, stress, or strain in the oars may be attached to a plurality of oars 160 in order to measure simultaneously the stress placed on a plurality of oars 160 by a plurality of athletes 110. The strain gauge 164, or other stress-measurement instrument, may be attached to the oar or may be embedded within the oar.

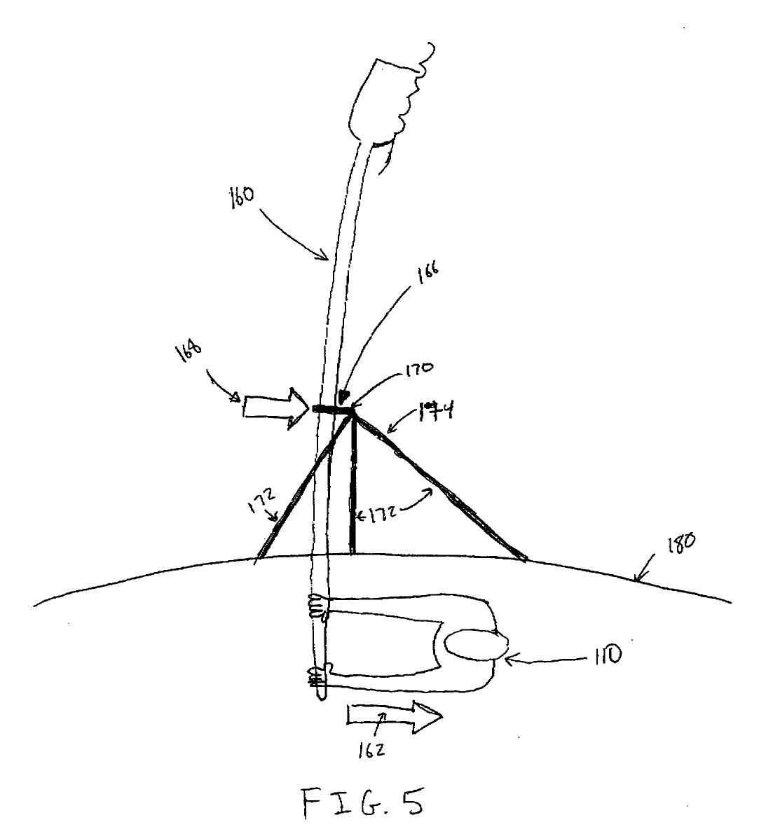

FIG. 5 is a schematic top view of a system used to monitor the load placed by the oar 160 on the boat. For example, the system may measure the load translated by the oar, not by the stress placed on the oar itself as in the previous paragraph, but by the force placed by the oar 160 against the point of contact 166 with the boat. The athlete 110 places load 162 against the oar 160, which translates directly to the oar 160 placing force 168 against the point of contact with the boat 166. The force 168 need not be defined as running parallel to the boat 180 or the forward direction of the boat and is defined as force placed against the boat, or extension thereof, from a plurality of angles. In a rowing boat, this point of contact 166 occurs where the oar 160 presses against the “oarlock” 170. The oarlock 170 is the part of the boat that encloses the oar 160, and can swivel. This contact point 166 occurs on/against the oarlock 170. Accordingly, the direction of force 168 will shift in direction as the athlete 110 moves the oar during a stroke. In some embodiments, a plurality off strain gauges, load cells, or other devices to measure force or load placed by the oar against the boat are used to measure the force at this point of contact 166, where the oar 160 presses against the oarlock 170. In some embodiments, load cells or other devices used to measure stress or load are placed in other components or areas of the rigger 172, or proximate areas from which load can be measured. The rigger 172 is an extension of the boat 180, and is defined as any structure providing indirect connection between the oar 160 and the boat 180. The term “boat” in this document refers to the boat 180 itself or any extension of the boat, such as the rigger 172, oarlock 170, or backstay 174 (which is a single piece component of the entire rigger unit 172). A strain gauge or other load/stress measurement device may be placed on any extension of the boat from which force transferred from the oar 160 could be measured. The strain gauges and load cells can be coupled to wireless transmitters on the boat or oars or by wires that run from the load cells or other load/stress measurement devices along the length of the boat or any extension of the boat. In some embodiments, a plurality of load cells or other force/stress measurement devices may be placed at a plurality of contact points 166 or integrated into a plurality of extensions of the boat, including, but not limited to, the rigger 172, the oarlock 170, the backstay 174, or the boat itself 180. The load cells may be connected to transmitters that can transmit the data to the receiver in real time. This would permit a simultaneous, real-time reading and display of the force placed by a plurality of oars against the boat or extension thereof at a plurality of positions, such as proximate to each athlete's 110 position in the boat. This would allow the system to measure the transfer of force from the athlete 110 to the forward movement of the boat.

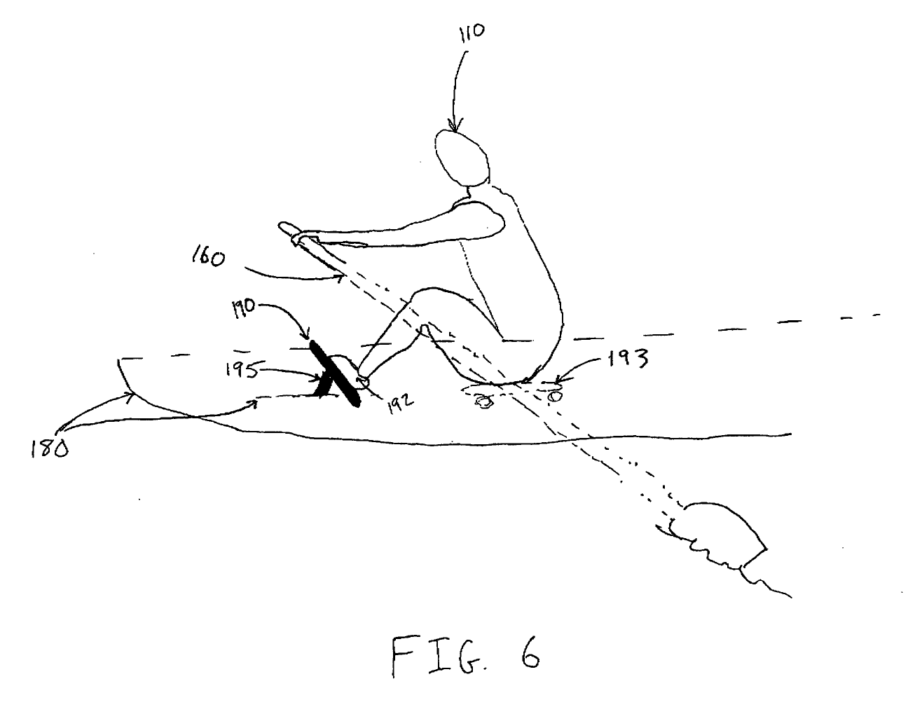

FIG. 6 is a schematic side view of an alternate means of measuring force output for an athlete rowing a boat 180. In some embodiments, the pressure placed by the legs of the athlete 110 on the foot-stretchers 190 translates the force placed by the athlete on the oar 160 as the force produced by the athlete extending the body is placed against both the oar 160 and the foot-stretcher 190. The seat 193 on which the athlete sits is able to move back and forth in the boat. The “foot-stretcher” refers to the part of the boat 180 where the athlete 110 attaches and presses his/her feet 192. In a rowing boat, the shoes are typically fixed to the boat 180 by a moveable piece known as the foot-stretcher 190 that affixes to the boat 180. In some embodiments, a load cell, or other instruments used for measuring load/stress, is affixed to or integrated into the foot-stretcher 190, or points of contact, or connecting parts 195, between the boat 180 and the foot-stretcher 190. The connecting mechanism 195 between the boat 180 and the foot-stretcher 190, or on the foot-stretcher 190, itself represents a possible, but not exclusive, point of placement for the load cell. In some embodiments, a load cell is placed proximate to the foot-stretcher 190 in a manner able to measure load or force placed against the foot-stretcher 190 by the athlete 110. In some embodiments, a plurality of load cells or other instruments used for measuring force/load are placed on or near a plurality of foot-stretchers 190 in order to simultaneously monitor the load/force placed by a plurality of athletes 110 against a respective plurality of foot-stretchers 190 at different locations in the boat 180.

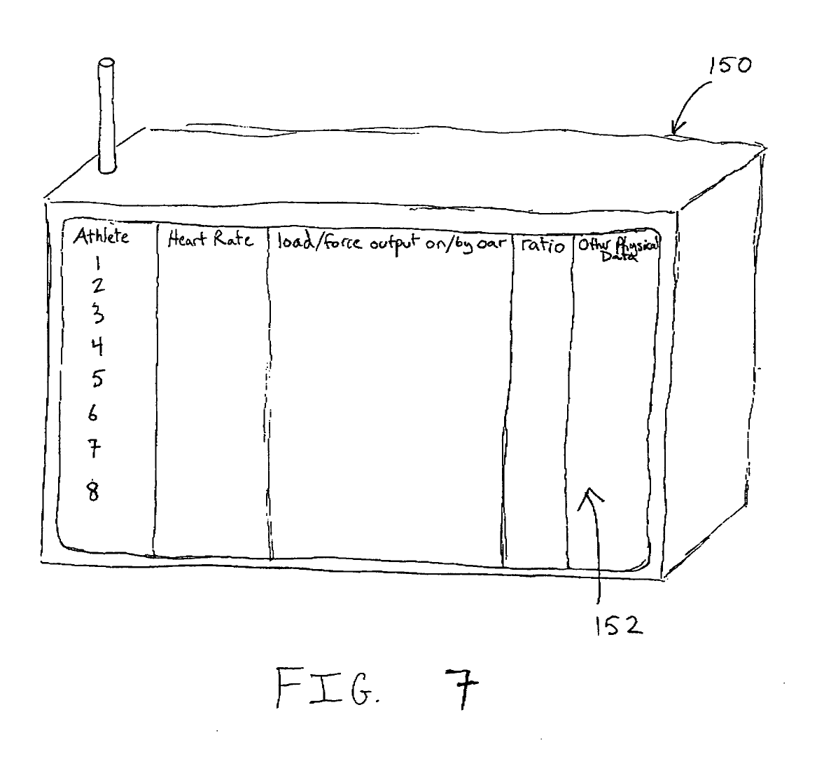

FIG. 7 shows an alternative embodiment of the display shown in FIG. 3. In some embodiments, the display 152 lists the force/load output of each athlete on the oar. In some embodiments, the display 152 lists force/load placed by the oar against the boat, or extensions of the boat, as defined above. In some embodiments, a plurality of data streams may be placed side-by-side on the display 152. In some embodiments, the display 152 lists a plurality of physical characteristics, such as heart rate, as well as force/load output data, side by side, for real-time comparison. In some embodiments, the display lists ratios or fractions of two different data streams. In some embodiments, the display 152 may have graphs, pie charts, or other graphical representations.

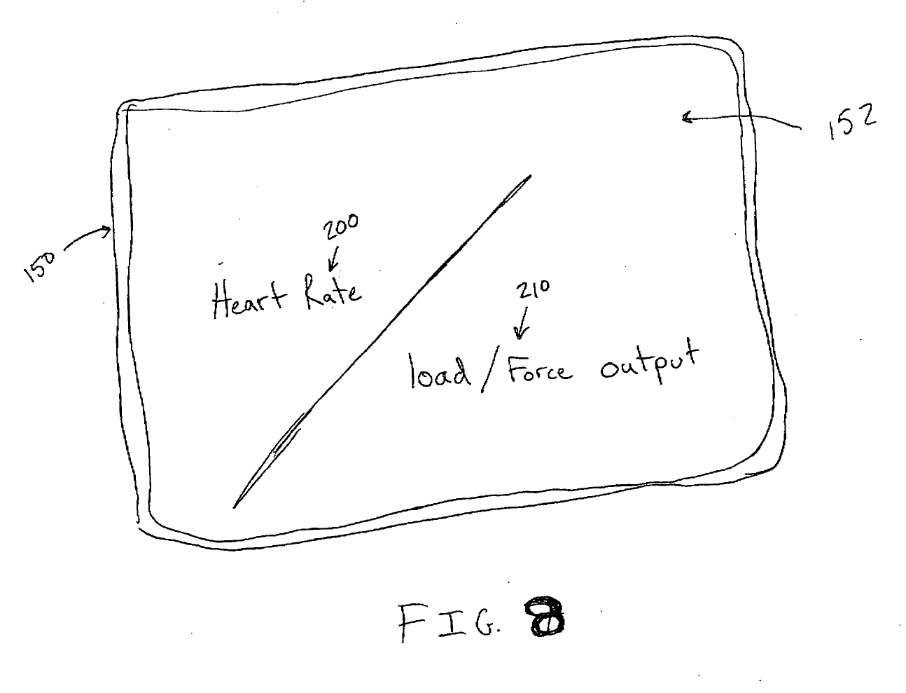

FIG. 8 is a schematic view of the display 152 of a key pairing of data, specifically a real-time heart rate reading 200 as compared to output/load/force data 210, as determined either by the stress placed on the oar by the athlete, or, by extension, the load placed by the oar against the boat, or an extension thereof, or the load placed by the feet of the athlete against the foot-stretcher.

Another embodiment of this aspect of the invention provides for monitoring at least one physical parameter of individuals engaging in a team sport. In some embodiments, the methods comprise the steps of (a) operatively coupling a plurality of sensors to a plurality of individuals engaging in a team sport, wherein each sensor is operatively coupled to one of the plurality of individuals, wherein each sensor is adapted to monitor and transmit information about at least one physical parameter of one of the plurality of individuals to a receiver adapted to receive and display information from the plurality of sensors, and wherein the physical parameter is at least one of a physiological parameter and a performance parameter; and (b) monitoring at least one physical parameter of each of the plurality of individuals by observing the information displayed on the receiver. The methods may further comprise the step of providing feedback to at least one of the plurality of individuals by recirculating the information provided to the display and transmitting this information back to an individual, verbally or visually, so that the individual may take action to alter the physiological parameters and performance parameters initially measured by the invention disclosed in this document.

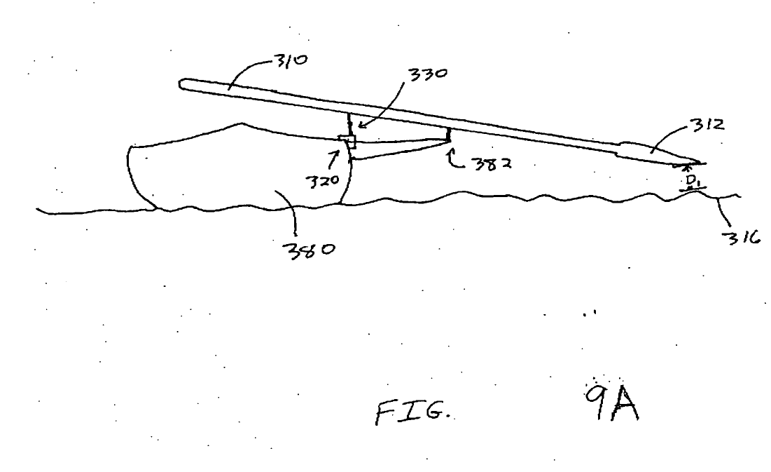

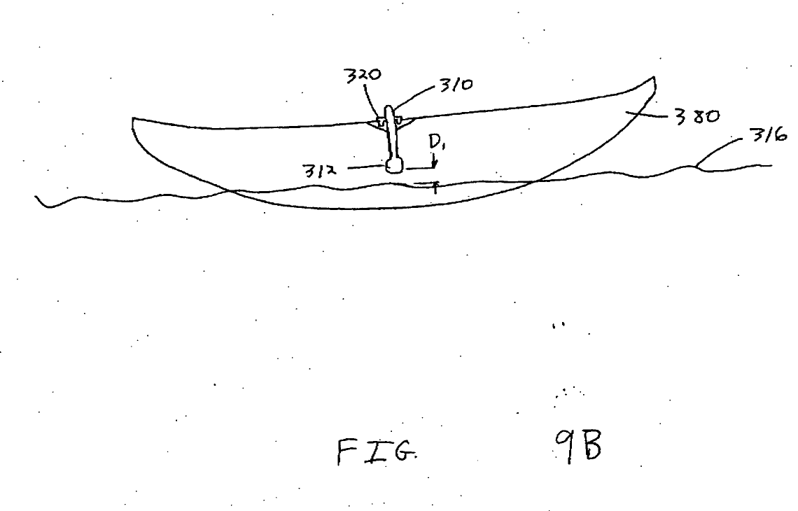

A second aspect of the invention provides apparatuses for indicating the position of an oar during rowing and methods for improving an individual's stroke technique by using an apparatus for indicating the position of an oar during rowing. For example, an apparatus for indicating the position of an oar may be used to indicate desirable oar positions. In some embodiments, the apparatus for indicating oar position comprises a position indicator extending from a support assembly operatively coupled to a boat. Exemplary embodiments of this aspect of the invention are illustrated in FIGS. 9-13.

FIGS. 9A and 9B are front and side views of a boat 380 having an oar 310 with a blade 312 and an outrigger 382 supporting the oar 310. The boat 380 also includes a position indicator 330 and a support assembly 320 that supports the position indicator 330. The position indicator 330 indicates when the blade 312 has reached or exceeded a predetermined maximum distance D1 from the water 316. Typically, the blade 312 is removed from the water 316 during the recovery portion of the rowing stroke. During recovery it is desirable to minimize the distance between the blade 312 and the water 316 to maximize the efficiency of the rowing process. In operation, the oar 310 contacts the position indicator 330 when the blade 312 has exceeded the predetermined maximum distance D1 from the water 316 thereby indicating an undesirable position of the blade 312. In some embodiments, the position indicator 330 is adjustable so that it can be positioned at different heights for different rowers. In other embodiments, the position indicator 330 may be fixed. In some embodiments, the indicator 330 may be struck by the oar handle and break away. In some embodiments, the indicator 330 may be flexible, allowing it to be struck by the oar handle, bending or deflecting away, and then returning to an upright position. Such an embodiment would allow a rower to strike the indicator 330 with the oar handle multiple times over the course of a practice, for example, with the indicator returning to an upright position between strokes. The oar indicator is used by the rower to practice clearing the oar handle at a specific, though variable, height by clearing the oar handle over the indicator 330. This allows for repetitive muscle-memory training over the course of many strokes and assists in improving the rower's stroke technique.

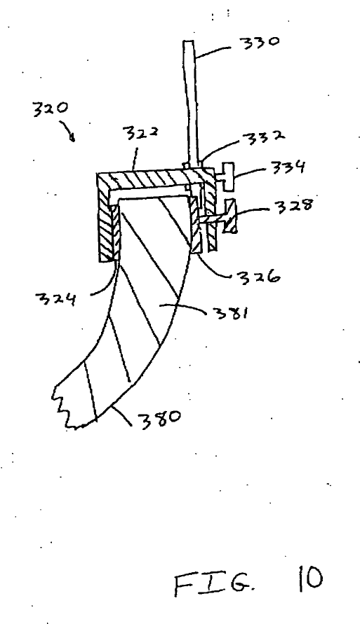

FIG. 10 is a cross-sectional front view of the support assembly 320 and the position indicator 330 of FIG. 9. The support assembly 320 is configured to engage a top portion of a sidewall 381 of the boat 380, and can include a support member 322, a first pad 324, a second pad 326, and a clamp fastener 328. The fastener 328 can be tightened to secure the support assembly 320 to the sidewall 381 of the boat 380. The pads 324 and 326 prevent the support assembly 320 from scratching or otherwise damaging the sidewall 381 of the boat 380. The support assembly 320 can also include a securing member 332 received in an aperture of the support member 322. The position indicator 330 is slidably received in the securing member 332 and a fastener 334, such as a clamp device, releasably secures the position indicator 330 in a desired position.

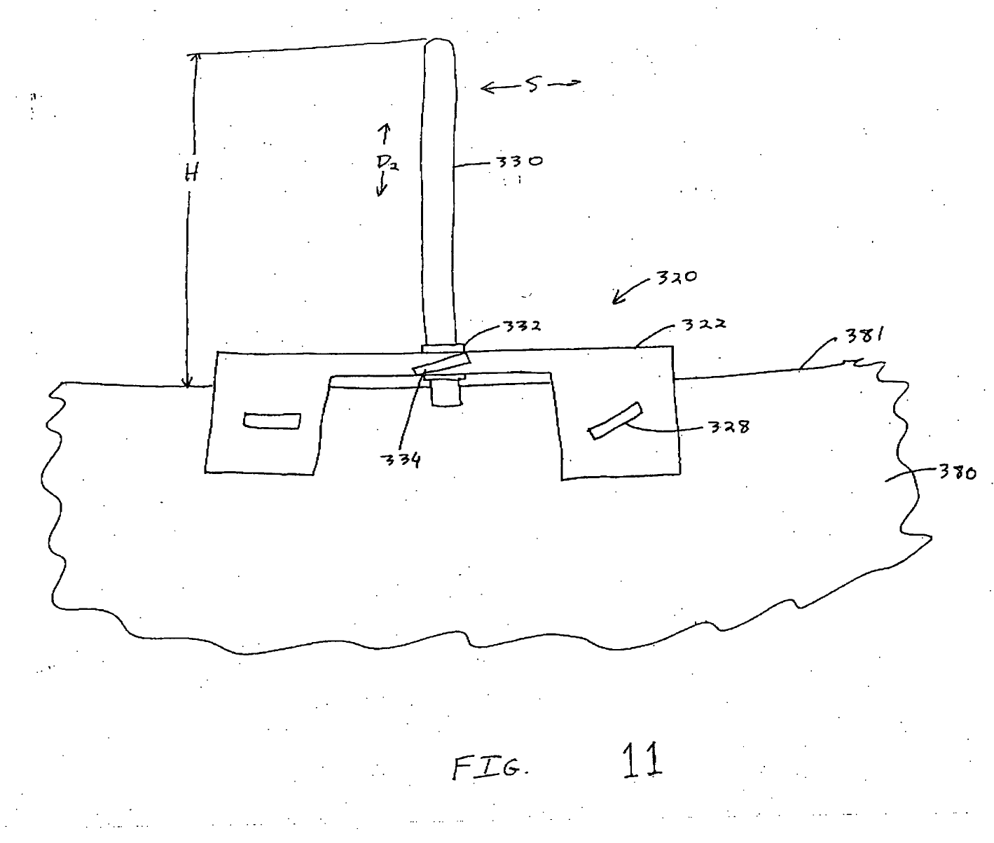

FIG. 11 is a side view of the support assembly 320 and the position indicator 330 of FIG. 10. The position indicator 330 can be moved in the upright direction D2 by adjusting the fastener 334 of the support assembly 320. Accordingly, the height H of the position indicator 330 can be set at a predetermined desired level. In the illustrated embodiment, the position indicator 330 is made of a flexible material so that when an oar strikes the position indicator 330, the top portion flexes in the direction S and thereafter returns to its original position. In some embodiments, the position indicator can include a wire, spring, or other material to allow it to move. In some embodiments, the position indicator may be rigid.

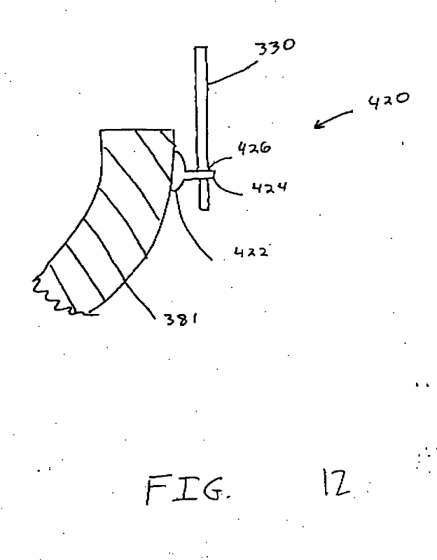

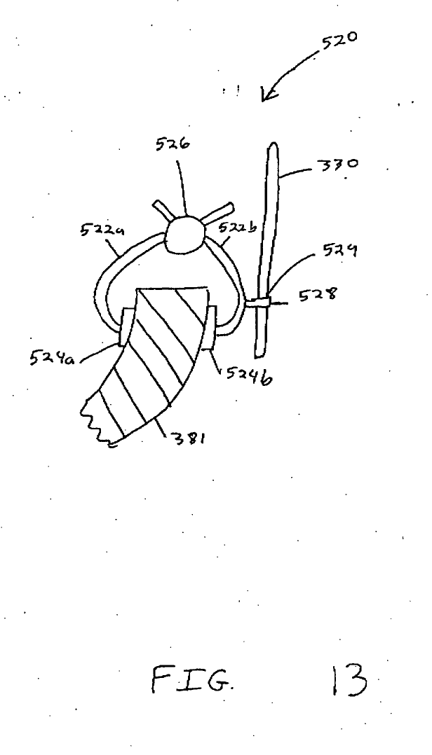

In some embodiments, several position indicators can be arranged on a sidewall of a boat. Such embodiments can be used, for example, to indicate the proper length of each rowing stroke. In some embodiments, the position indicator 330 may have other configurations. For example, the position indicator 330 may include two vertical members and a horizontal member extending between the two vertical members. In some embodiments, the support assembly 320 can have other configurations, such as those described below with reference to FIGS. 12 and 13.

FIG. 12 is a fragmentary cross-sectional front view of a support assembly 420 in accordance with some embodiments of the invention. The support assembly 420 can include a coupling member 422 coupled to the sidewall 381 of the boat 380. The coupling member 422 can be a suction cup or other suitable device. The support assembly 420 also includes a projection 424 coupled to the coupling member 422. The projection 424 includes an aperture 426 to receive the position indicator 330.

FIG. 13 is a fragmentary cross-sectional front view of a support assembly 520 in accordance with some embodiments of the invention. The support assembly 520 includes a first curved arm 522a, a second curved arm 522b, and a spring 526 operably coupling the first arm 522a to the second arm 522b. The support assembly 520 also includes a first pad 524a coupled to the end of the first arm 522a and a second pad 524b coupled to the end of the second arm 522b. Accordingly, the spring 526 urges the first and second arms 522a-b and the first and second pads 524a-b to engage the sidewall 381 of the boat 380. The support assembly 520 also includes a transverse support member 528 coupled to the second arm 522b that has an aperture 529 to receive the position indicator 330. In some embodiments, the support assembly can have other configurations.

The description of the embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teaching of the invention provided herein can be applied to other systems, not only the system described herein. The various embodiments described herein can be combined to provided further embodiments. Thus, while the preferred embodiments of the invention have been illustrated and described, it will be appreciated that various changes can be made therein without departing from the spirit and scope of the invention.

1. A system for monitoring at least one physical parameter of individuals engaging in a team sport, the system comprising:
(a) a plurality of sensors, wherein each sensor is operatively coupled to one of a plurality of individuals engaging in a team sport, wherein each sensor is adapted to monitor and transmit information about at least one physical parameter of one of the plurality of individuals, and wherein the physical parameter is at least one of a physiological parameter and a performance parameter; and
(b) a receiver adapted to receive and display the transmitted information from each of the plurality of sensors.

2. The system of claim 1, wherein the team sport is rowing.

3. The system of claim 1, wherein the plurality of sensors comprises sensors adapted for monitoring a physiological parameter selected from the group consisting of heart rate, temperature, breathing rate, perspiration rate, blood pressure, and lactic acid levels.

4. The system of claim 2, wherein the plurality of sensors comprises sensors adapted to monitor a performance parameter selected from the group consisting of stress placed on an oar by an individual, load placed on an oar against a boat, and load placed on a boat by an individual.

5. The system of claim 1, wherein the plurality of sensors comprises sensors adapted to monitor a performance parameter and sensors adapted for monitoring a physiological parameter from each of the plurality of individuals.

6. A method for monitoring at least one physical parameter of individuals engaging in a team sport, comprising the steps of:
(a) operatively coupling a plurality of sensors to a plurality of individuals engaging in a team sport, wherein each sensor is operatively coupled to one of the plurality of individuals, wherein each sensor is adapted to monitor and transmit information about at least one physical parameter of one of the plurality of individuals to a receiver adapted to receive and display information from the plurality of sensors, and wherein the physical parameter is at least one of a physiological parameter and a performance parameter; and
(b) monitoring at least one physical parameter of each of the plurality of individuals by observing the information displayed on the receiver.

7. The method of claim 6, further comprising the step of providing feedback to at least one of the plurality of individuals.
